(19) United States
(12) Patent Application Publication
(10) Pub. No.: US 2014/0079775 A1
(43) Pub. Date: Mar. 20, 2014

(54) AGENTS TO TREAT/PREVENT AMOEBIASIS

(71) Applicant: The Regents of the University of California, Oakland, CA (US)

(72) Inventors: Lars Bode, San Diego, CA (US); Tineke Lauwaet, San Diego, CA (US); Evelyn Jantscher-Krenn, San Diego, CA (US); Frances Gillin, La Jolla, CA (US)

(73) Assignee: The Regents of the University of California, Oakland, CA (US)

(21) Appl. No.: 13/983,746

(22) PCT Filed: Feb. 3, 2012
(86) PCT No.: PCT/US12/23863

§ 371 Date: Nov. 20, 2013

Related U.S. Application Data
(60) Provisional application No. 61/439,749, filed on Feb. 4, 2011.

(51) Int. Cl. A61K 31/702; A23L 1/30

(52) U.S. Cl. CPC A61K 31/702; A23L 1/30
USPC 424/451; 514/59; 424/464

(57) ABSTRACT

The invention provides formulations comprising isolated lacto-N-tetraose (LNT) or galactooligosaccharides (GOS) or variants, isomers, analogs and derivatives thereof and a pharmaceutically acceptable carrier.

Throughout this application various publications are referenced. The disclosures of these publications in their entirety are hereby incorporated by reference into this application in order to more fully describe the state of the art to which this invention pertains.

### BACKGROUND OF THE INVENTION

Entamoeba histolytica, is a human enteric protozoan parasite that causes amoebic colitis. In developing countries, high incidence and relapse rates pose a severe health threat especially for infants and young children, among whom infectious diarrhea is the main cause of morbidity and mortality. With currently no vaccine available and chemotherapeutics exerting strong side effects, there is a high need of a safe, preventive and therapeutic treatment. Breastfeeding has been shown to be the best intervention in infants to reduce the incidence of infectious diseases, such as amoebiasis.

The protozoan parasite Entamoeba is highly prevalent in developing countries, especially in areas with poor sanitary conditions. The pathogenic Entamoeba histolytica is microscopically indistinguishable from the more common non-pathogenic Entamoeba dispar and E. moshkovskii. The latter species are commensals and do not require treatment. However, infection with E. histolytica can lead to invasive amoebiasis with hematogenous spread to the liver, lungs and brain and cause life-threatening conditions and must be treated.

PCR is currently the most accurate way to identify the Entamoeba isolate but is expensive and therefore not always available in endemic areas. Fast and inexpensive ‘dipstick’ detection kits would be ideal and are currently under development.

Once infection with E. histolytica has been confirmed, patients are treated according to their clinical manifestations. Asymptomatic patients are treated with the luminal amebicides paromomycin or diloxanide furoate. These agents eliminate luminal ameba and prevent invasive disease and formation of infectious cysts.

Symptomatic patients with intestinal and/or extraintestinal invasive amoebiasis are most commonly treated with 5-nitroimidazoles (e.g. metronidazole) followed by a luminal agent to eliminate luminal parasites.

The most common side effects of these drugs are abdominal discomfort and nausea. Serious adverse effects of metronidazole include confusion, ataxia and seizure.

The use of amebicides to prevent amoebiasis might lead to drug resistance and is not recommended. The effectiveness of vaccines is currently being studied, but is not yet developed. Therefore, other approaches are needed.

### SUMMARY OF THE INVENTION

The invention provides formulations for a subject comprising isolated lacto-N-tetraose (LNT) and/or galactooligosaccharides (GOS) or its variants, isomers, analogs and derivatives thereof and a pharmaceutically acceptable carrier.

The invention also provides methods to prevent or treat a disease or disorder in a subject by administering human milk oligosaccharides (HMO) or galactooligosaccharides (GOS), or equivalents, analogs and derivatives thereof in an amount sufficient to treat and prevent the disease or disorder. In one embodiment, the HMO is an isolated lacto-N-tetraose (LNT).

### BRIEF DESCRIPTION OF THE FIGURES

FIG. 1 HMO detach E. histolytica trophozoites in a reversible manner. Representative HPLC chromatogram of pooled HMO (A). Attached HM-1 trophozoites were incubated with different HMO concentrations for 30 min at 37° C. (B) or with 2.5 or 10 mg/ml for different periods of time (C). Detached and attached cells were counted and the percentage of detached cells was calculated and normalized to the untreated control. Data points and flags represent the means±SEM of 3 independent experiments performed in duplicate. Reversibility was assessed by removing HMO and allowing detached trophozoites to reattach (D). Detached and re-attached trophozoites are expressed as the percentage of total trophozoites (attached+detached).

FIG. 2 HMO prevent E. histolytica mediated cytotoxicity in in vitro co-culture with enterocytes. Representative images of methylene blue stained HT-29 cell layers as such (A, left) or cocultured with HM-1 trophozoites in the absence (A, middle) or presence (A, right) of 10 mg/ml HMO. Dose response curve with different concentrations of HMO (B). The O.D 660 nm of methyleneblue cell layers was used as a measure for cytotoxicity. The cytoprotective effect of HMO was calculated as the percent ‘cell rescue’ compared to the untreated coculture (0%). Results are means SEM of 4 independent experiments. Cocultures were incubated with different glycans and cytotoxicity was determined as described above (C). 10 mM galactose, glucose and fucose have no significant effect while 10 mM Lactose and 20 mg/ml HMO significantly prevent cytotoxicity. HMO rescues cell layers when added to existing cocultures (D). HT-29 cells were cocultured with HM-1 and 20 mg/ml HMO was added at the indicated times of coculture. Cocultures were stopped and cytotoxicity was determined after 2 hrs. Control cell layers were cocultured for 2 hrs in the absence of HMO and were considered to have 0% cell rescue.

FIG. 3 Protection of the HT-29 monolayer by individual HMO. Cell rescue in co-culture treated with physiologically relevant concentrations of individual HMO (A). Symbols represent structures of individual HMO. Cell survival in the presence of equimolar concentrations of LNT, LNFP1 and a mixture of LNFP1, LNFP2, and LNFP3. LNT has significantly more effect than LNFP1 and LNFP1, 2, 3 (indicated by stars)(B). Cytotoxicity assay with 10 mM 2′FL before and after fucosidase digestion (C). 2′FL left untreated or incubated with alpha1, 2 fucosidase was used in cytotoxicity assay. As controls, we used enzyme reaction buffer with inactivated fucosidase (control); equimolar concentrations (10 mM) of Lac, Fuc, or Fuc and Lac combined. Data from cytotoxicity assays are presented as % cell rescue compared to untreated cocultures (0%). Error bars are SEM, stars indicate statistical significance compared to control unless indicated by bars.

FIG. 4 GOS reduces cytotoxicity of trophozoites independent of its lactose content. Cell rescue in cocultures treated with increasing concentrations of GOS (triangles) or its corresponding lactose content (circles)(A). Cytotoxicity assay with GOS fractions (B). Higher numbered (eluted later) fractions contain lower molecular weight carbohydrates. The lactose content of fractions 11 and 12 is indicated above the respective bars. HPLC chromatogram of the original GOS preparation (20% lactose) (upper panel) and of the lactose free GOS (<0.5%) (C). Cytotoxicity assay with increasing concentrations of lactose-free GOS (triangles) and Lac-containing GOS (circles)(D).

### DETAILED DESCRIPTION OF THE INVENTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety.

As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b. Unless otherwise indicated, all numbers expressing quantities of ingredients, properties such as molecular weight, reaction conditions, and so forth as used herein, are to be understood as being modified in all instances by the term “about.” Accordingly, unless indicated to the contrary, the numerical parameters herein are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

As used herein, the terms “subject” and “patient” refer to any animal, such as a mammal. Mammals include but are not limited to, humans, murines, simians, felines, canines, equines, bovines, porcines, ovines, caprines, rabbits, mammalian farm animals, mammalian sport animals, and mammalian pets. In many embodiments, the hosts will be humans.

Isolated LNT and/or GOS and/or its variant, isomer, analog and/or derivative may be obtained by purifying LNT from nature or synthesized using known chemical or biochemical principles and methods. As used herein, the term “isolated” in reference to LNT and/or GOS of the invention does not require absolute purity.

The invention provides formulations comprising isolated lacto-N-tetraose (LNT) and/or galactooligosaccharides (GOS) or its variants, isomers, and derivatives thereof and a pharmaceutically acceptable carrier.

In one embodiment, the formulation is a pharmaceutical formulation. In one embodiment, the pharmaceutical formulation is an infant formula, baby food or nutritional supplement. In another embodiment, the subject is a human, monkey, rat, mouse, dog, cat, pig, goat, sheep, horse or cow.

The invention also provides methods to prevent or treat a disease or disorder in a subject by administering human milk oligosaccharides (HMO) or galactooligosaccharides (GOS), or equivalents, analogs and derivatives thereof in an amount sufficient to treat and prevent the disease or disorder. In one embodiment, the HMO is an isolated lacto-N-tetraose (LNT).

In another embodiment, the subject is a human, monkey, rat, mouse, dog, cat, pig, goat, sheep, horse or cow.

In one embodiment, the formulation provides the human HMO or GOS or variants, isomers, analogs and derivatives thereof in an amount sufficient to inhibit a disease or disorder (including but not limited to a parasitic disease, gastrointestinal disease, amoebiasis, amoebic dysentery or amoebic colitis infection).

Further, the amount sufficient to inhibit the disease or disorder may be about is at least greater than 700 μM, greater than 1400 μM, greater than 15,000 μM, or in the range of about 20,000-25,000 μM or 140-28,000 μM. Other amounts are possible.

In accordance with the practice of the invention, the formulation may be an enteral formulation.

Further the formulation of the invention may be included or added to in an infant formula, water, juices, breast milk, baby food. In some embodiments, the formulation is a nutritional supplement.

In one embodiment, the formulation is a tablet or a caplet. In another embodiment, the tablet or caplet is multi-layered. In another embodiment, the tablet or caplet is a matrix tablet or caplet.

In one embodiment, the formulation is a multiparticulate formulation. In another embodiment, the multiparticulates are encapsulated. In yet another embodiment, the multiparticulates are pressed into a tablet.

In one embodiment, the disease or disorder is a parasitic disease, gastrointestinal disease, amoebiasis, amoebic dysentery or amoebic colitis. In another embodiment, the disease is associated with Entamoeba histolytica infection.

In one embodiment, the human milk oligosaccharides (HMO) and/or galactooligosaccharides (GOS), or their equivalents, analogs and derivatives may be administered by means of infant formula, fortified breast milk, baby food, biscuits, adult nutritional supplements, dairy products, fruit drinks, acidic beverages, confectionery, cereal bars or baked goods.

The present invention provides pharmaceutical formulations (also known as pharmaceutical compositions or dosage forms) comprising isolated LNT and/or GOS, and a pharmaceutically acceptable carrier or vehicle.

Pharmaceutically acceptable carrier or vehicle refers to a non-toxic solid, semisolid (also referred to herein as softgel) or liquid filler, diluent, encapsulating material or formulation auxiliary of any type. The invention also provides methods for treating or ameliorating amoebiasis (and other parasitic infections) using said pharmaceutical formulations.

Further, isolated LNT and/or GOS of the invention can be pegylated, phosphorylated, esterified, derivatized with amino acids and/or peptides, to improve solubility for both formulation and bioavailability. Additionally, lipid derivatization and other lipophile derivatization can be used to improve mucosal permeability, absorption and formulation.

Dosage forms can be made according to well known methods in the art.

The pharmaceutical compositions of the invention may be formulated as solid dosage forms, such as capsules, pills, softgels, tablets, caplets, troches, wafer, sprinkle, chewing gum or the like, for oral administration. The pharmaceutical compositions of the invention may also be formulated as liquid dosage forms such as elixir, suspension or syrup.

The pharmaceutical compositions of the invention may also be presented in a dosage form for transdermal application, for example an ointment for children, a form for oral administration, for example a slow release product, or in gastro-resistant tablet form or gum form. They may also be in spray, bronchial form or eye lotion form, or other galenic forms with programmed mucosal and secondarily per os disintegration.

Therefore the different pharmaceutical compositions of the invention can be administered by several routes chosen in accordance with the patient's pathological profile and age. For children, the patch form, syrup form or tablets to be dissolved in the mouth. The other forms, eye lotion or injection may also be used. In adults all galenic forms (also known as dosage forms) can be contemplated.

The advantage of a coupled or combined galenic form also provides simplicity of treatment, patient compliance with the simplified treatment and therefore a more successful outcome.

The pharmaceutical compositions of the present invention may be mixed with pharmaceutically acceptable carriers, binders, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, coloring agent, dyes, preservatives and dispensing agents, or compounds of a similar nature depending on the nature of the mode of administration and dosage forms. Such ingredients, including pharmaceutically acceptable carriers and excipients that may be used to formulate oral dosage forms, are described in the Handbook of Pharmaceutical Excipients, American Pharmaceutical Association (1986), incorporated herein by reference in its entirety.

Pharmaceutically acceptable carriers are generally non-toxic to recipients at the dosages and concentrations employed and are compatible with other ingredients of the formulation. Examples of pharmaceutically acceptable carriers include water, saline, Ringer's solution, dextrose solution, ethanol, polyols, vegetable oils, fats, ethyl oleate, liposomes, waxes polymers, including gel forming and non-gel forming polymers, and suitable mixtures thereof. The carrier may contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulin; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG. Preferably the carrier is a parenteral carrier, more preferably a solution that is isotonic with the blood of the recipient.

Examples of binders include, but are not limited to, microcrystalline cellulose and cellulose derivatives, gum tragacanth, glucose solution, acacia mucilage, gelatin solution, molasses, polvinylpyrrolidine, povidone, crospovidones, sucrose and starch paste.

Examples of diluents include, but are not limited to, lactose, sucrose, starch, kaolin, salt, mannitol and dicalcium phosphate.

Examples of excipients include, but are not limited to, starch, surfactants, lipophilic vehicles, hydrophobic vehicles, pregelatinized starch, Avicel, lactose, milk sugar, sodium citrate, calcium carbonate, dicalcium phosphate, and lake blend purple. Typical excipients for dosage forms such as a softgel include gelatin for the capsule and oils such as soy oil, rice bran oil, canola oil, olive oil, corn oil, and other similar oils; glycerol, polyethylene glycol liquids, vitamin E TPGS as a surfactant and absorption enhancer (Softgels: Manufacturing Considerations; Wilkinson P, Foo Sog Hom, Special Drug Delivery Systems; Drugs and the Pharmaceutical Sciences Vol 41 Praveen Tyle Editor, Marcel Dekker 1990, 409-449; Pharmaceutical Dosage Rums and Drug Delivery by Ansel, Popovich and Allen 1995, Williams and Wilkins, Chapter 5 pp 155-225).

Examples of disintegrating agents include, but are not limited to, complex silicates, croscarmellose sodium, sodium starch glycolate, alginic acid, corn starch, potato starch, bentonite, methylcellulose, agar and carboxymethylcellulose.

Examples of glidants include, but are not limited to, colloidal silicon dioxide, talc, corn starch.

Examples of wetting agents include, but are not limited to, propylene glycol monostearate, sorbitan mono oleate, diethylene glycol monolaurate and polyoxyethylene laural ether.

Examples of sweetening agents include, but are not limited to, sucrose, lactose, mannitol and artificial sweetening agents such as saccharin, and any number of spray dried flavors.

Examples of flavoring agents include, but are not limited to, natural flavors extracted from plants such as fruits and synthetic blends of compounds which produce a pleasant sensation, such as, but not limited to peppermint and methyl salicylate.

Examples of lubricants include magnesium or calcium stearate, sodium lauryl sulphate, talc, starch, lycopodium and stearic acid as well as high molecular weight polyethylene glycols.

Examples of coloring agents include, but are not limited to, any of the approved certified water soluble FD and C dyes, mixtures thereof; and water insoluble FD and C dyes suspended on alumina hydrate.

The artisan of ordinary skill in the art will recognize that many different ingredients can be used in formulations according to the present invention, in addition to the active agents (an HMO or GOS), while maintaining effectiveness of the formulations in treating e.g., amoebosis. The list provided herein is not exhaustive.

### Matrix Based Dosage Forms

Dosage forms according to one embodiment of the present invention may be in the form of coated or uncoated matrices. The term matrix, as used herein, is given its well-known meaning in the pharmaceutical arts as a solid material having an active agent (e.g., the components of the compositions of the invention) of the invention incorporated therein. Upon exposure to a dissolution media, channels are formed in the solid material so that the active agent can escape.

The skilled artisan will appreciate that the matrix material can be chosen from a wide variety of materials which can provide the desired dissolution profiles. Materials can include, for example, one or more gel forming polymers such as polyvinyl alcohol, cellulose ethers including, for example, hydroxypropylalkyl celluloses such as hydroxypropyl cellulose, hypromellose, prop-2-enoic acid, hydroxypropyl methyl cellulose, hydroxyalkyl celluloses such as hydroxypropyl cellulose, natural or synthetic gums such as guar gum, xanthum gum, and alginates, as well as ethyl cellulose, polyvinyl pyrrolidone, fats, waxes, polycarboxylic acids or esters such as the Carbopol R series of polymers, methacrylic acid copolymers, and methacrylate polymers.

In addition to the above-mentioned ingredients, a controlled release matrix may also contain suitable quantities of other materials, for example, diluents, lubricants, binders, granulating aids, colorants, flavorants, and glidants that are conventional in the pharmaceutical arts. The quantities of these additional materials should be sufficient to provide the desired effect to the desired formulation. A controlled release matrix incorporating particles may also contain suitable quantities of these other materials such as diluents, lubricants, binders, granulating aids, colorants, flavorants, and glidants that are conventional in the pharmaceutical arts in amounts up to about 75% by weight of the particulate, if desired.

Methods of making matrix dosages are well known in the art and any known method of making such dosages which yields the desired immediate release and controlled release dissolution profiles can be used. One such method involves the mixture of the compositions of the invention with a solid polymeric material and one or more pharmaceutically acceptable excipients which can then be blended and compressed in controlled release tablet cores. Such tablet cores can be used for further processing as bi-layer or multilayer tablets, press coated tablets, or film coated tablets.

In addition, the formulation of respective release components can occur by appropriate granulation methods as is well known in the art. In wet granulation, solutions of the binding agent can be added with stirring to the mixed powders. The powder mass can be wetted with the binding solution until the mass has the consistency of damp snow or brown sugar. The wet granulated material can be forced through a sieving device. Moist material from the milling step can be dried by placing it in a temperature controlled container. After drying, the granulated material can be reduced in particle size by passing it through a sieving device. Lubricant can be added, and the final blend can then be compressed into a matrix dosage form such as a matrix tablet.

In fluid-bed granulation, particles of inert material and/or active agent (e.g., the components of the compositions of the invention) can be suspended in a vertical column with a rising air stream. While the particles are suspended, a common granulating material in solution can be sprayed into the column. There will be a gradual particle buildup under a controlled set of conditions resulting in tablet granulation. Following drying and the addition of lubricant, the granulated material will be ready for compression.

In dry-granulation, the active agent (e.g., the components of the compositions of the invention), binder, diluent, and lubricant can be blended and compressed into tablets. The compressed large tablets can be comminuted through the desirable mesh screen by sieving equipment. Additional lubricant can be added to the granulated material and blended gently. The material can then be compressed into tablets.

### Particle Based Dosage Forms

### Immediate Release and Controlled Release Particles

Dosage forms according to another embodiment of the present invention may be in the form of coated or uncoated immediate release/controlled release dosage forms. The immediate release/controlled release dosage forms of the present invention can take the form of pharmaceutical particles. The dosage forms can include immediate release particles in combination with controlled release particles in a ratio sufficient to deliver the desired dosages of active agents (e.g., the components of the compositions of the invention). The controlled release particles can be produced by coating the immediate release particles with an enteric coat.

The particles can be produced according to any of a number of well-known methods for making particles. The immediate release particles can comprise the active agent combination (the compositions of the invention) and a disintegrant. Suitable disintegrants can include, for example, starch, low-substitution hydroxypropyl cellulose, croscarmellose sodium, calcium carboxymethyl cellulose, hydroxypropyl starch, and microcrystalline cellulose.

In addition to the above-mentioned ingredients, a controlled release matrix may also contain suitable quantities of other materials, for example, diluents, lubricants, binders, granulating aids, colorants, flavorants, and glidants that are conventional in the pharmaceutical arts. The quantities of these additional materials should be sufficient to provide the desired effect to the desired formulation. A controlled release matrix incorporating particles may also contain suitable quantities of these other materials such as diluents, lubricants, binders, granulating aids, colorants, flavorants, and glidants that are conventional in the pharmaceutical arts in amounts up to about 75% by weight of the particulate, if desired.

Particles can assume any standard structure known in the pharmaceutical arts. Such structures can include, for example, matrix particles, non-pareil cores having a drug layer and active or inactive cores having multiple layers thereon. A controlled release coating can be added to any of these structures to create a controlled release particle.

The term particle as used herein means a granule having a diameter of between about 0.01 mm and about 5.0 mm, preferably between about 0.1 mm and about 2.5 mm, and more preferably between about 0.5 mm and about 2 mm. The skilled artisan will appreciate that particles according to the present invention can be any geometrical shape within this size range and so long as the mean for a statistical distribution of particles falls within the particle sizes enumerated above, they will be considered to fall within the contemplated scope of the present invention.

The release of the therapeutically active agent (e.g., the components of the compositions of the invention) from the controlled release formulation of the present invention can be further influenced, i.e., adjusted to a desired rate, by the addition of one or more release-modifying agents. The release-modifying agent may be organic or inorganic and include materials that can be dissolved, extracted, or leached from the coating in the environment of use. The pore-formers may comprise one or more hydrophilic materials such as hydroxypropyl methylcellulose. The release-modifying agent may also comprise a semi-permeable polymer. In certain preferred embodiments, the release-modifying agent is selected from hydroxypropyl methylcellulose, lactose, metal stearates, and mixtures thereof.

The controlled release particles of the present invention can slowly release the compositions of the invention when ingested. The controlled release profile of the formulations of the present invention can be altered, for example, by increasing or decreasing the thickness of a retardant coating, i.e., by varying the amount of overcoating. The resultant solid controlled release particles may thereafter be placed in a gelatin capsule in an amount sufficient to provide an effective controlled release dose when ingested and contacted by an environmental fluid, e.g., gastric fluid, intestinal fluid or dissolution media.

The dosage forms of the invention may be coated (e.g., film coated or enterically coated) as known by those of skill in the art. For example, the composition can be formulated in an enteric coating that maintains its integrity in the stomach and releases the active compound in the intestine.

Examples of enteric-coatings include, but are not limited to, phenylsalicylate, fatty acids, fats, waxes, shellac, ammoniated shellac and cellulose acetate phthalates. Film coatings include, but are not limited to, hydroxyethylcellulose, sodium carboxymethylcellulose, polyethylene glycol 4000 and cellulose acetate phthalate.

In one example, the dosage forms e.g., particles of the invention as described above, may be overcoated with an aqueous dispersion of a hydrophobic or hydrophilic material to modify the release profile. The aqueous dispersion of hydrophobic material preferably further includes an effective amount of plasticizer, e.g. triethyl citrate. Preformulated aqueous dispersions of ethylcellulose, such as AQUACOAT™ or SURELEASE™ products, may be used. If a SURELEASE™ product is used, it is not necessary to separately add a plasticizer.

The hydrophobic material may be selected from the group consisting of alkylcellulose, acrylic and methacrylic acid polymers and copolymers, shellac, zein, fatty oils, hydrogenated castor oil, hydrogenated vegetable oil, or mixtures thereof. In certain preferred embodiments, the hydrophobic material can be a pharmaceutically acceptable acrylic polymer including, but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid anhydride), polymethacrylate, polyacrylamide, poly(methacrylic acid anhydride), and glycidyl methacrylate copolymers. In alternate embodiments, the hydrophobic material can be selected from materials such as one or more hydroxyalkyl celluloses such as hydroxypropyl methylcellulose. The hydroxyalkyl cellulose can preferably be a hydroxy (C.sub.1 to C.sub.6) alkyl cellulose, such as hydroxypropylcellulose, hydroxypropylmethylcellulose, or preferably hydroxyethylcellulose. The amount of the hydroxyalkyl cellulose in the present oral dosage form can be determined, in part, by the precise rate of active agents (e.g., the components of the compositions of the invention) desired and may vary from about 1% to about 80%.

In embodiments of the present invention where the coating comprises an aqueous dispersion of a hydrophobic polymer, the inclusion of an effective amount of a plasticizer in the aqueous dispersion of hydrophobic polymer can further improve the physical properties of the film. For example, because ethylcellulose has a relatively high glass transition temperature and does not form flexible films under normal coating conditions, it may be necessary to plasticize the ethylcellulose before using it as a coating material. Generally, the amount of plasticizer included in a coating solution can be based on the concentration of the film-former, e.g., most often from about 1 percent to about 50 percent by weight of the film-former. Concentration of the plasticizer, however, can be preferably determined after careful experimentation with the particular coating solution and method of application.

Examples of suitable plasticizers for ethylcellulose include water-insoluble plasticizers such as dibutyl sebacate, diethyl phthalate, triethyl citrate, tributyl citrate, and triacetin, although other water-insoluble plasticizers (such as acetylated monoglycerides, phthalate esters, castor oil, etc.) may be used. Triethyl citrate may be an especially preferred plasticizer for the aqueous dispersions of ethyl cellulose of the present invention.

Examples of suitable plasticizers include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as EUDRAGIT™ RL/RS lacquer solutions include polyethylene glycols, propylene glycol, diethyl phthalate, castor oil, and triacetin. Triethyl citrate may be an especially preferred plasticizer for aqueous dispersions of ethyl cellulose. It has further been found that addition of a small amount of talc may reduce the tendency of the aqueous dispersion to stick during processing and acts a polishing agent.

One commercially available aqueous dispersion of ethylcellulose is the AQUACOAT™ product which is prepared by dissolving the ethylcellulose in a water-immiscible organic solvent and then emulsifying the ethylcellulose in water in the presence of a surfactant and a stabilizer. After homogenization to generate submicron droplets, the organic solvent can be evaporated under vacuum to form a pseudolatex. The plasticizer will not be incorporated into the pseudolatex during the manufacturing phase. Thus, prior to using the pseudolatex as a coating, the AQUACOAT™ product can be mixed with a suitable plasticizer.

Another aqueous dispersion of ethylcellulose is commercially available as SURELEASE™ product (Colorcon, Inc., West Point, Pa., U.S.A.). This product can be prepared by incorporating plasticizer into the dispersion during the manufacturing process. A hot melt of a polymer, plasticizer (dibutyl sebacate), and stabilizer (oleic acid) can be prepared as a homogeneous mixture which can then be diluted with an alkaline solution to obtain an aqueous dispersion which can be applied directly onto substrates.

In one embodiment, the acrylic coating can be an acrylic resin lacquer used in the form of an aqueous dispersion, such as that which is commercially available from Rohm Pharma under the trade name EUDRAGIT™. In additional embodiments, the acrylic coating can comprise a mixture of two acrylic resin lacquers commercially available from Rohm Pharma under the trade names EUDRAGIT™ RL 30 D and EUDRAGIT™ RS 30 D. EUDRAGIT™ RL 30 D and EUDRAGIT™ RS 30 are copolymers of acrylic and methacrylic esters with a low content of quaternary ammonium groups, the molar ratio of ammonium groups to the remaining neutral (meth)acrylic esters being 1:20 in EUDRAGIT™ RL 30 and 1:40 in EUDRAGIT™ RS 30 D. The mean molecular weight is about 150,000 Daltons. The code designations RL (high permeability) and RS (low permeability) refer to the permeability properties of these agents. EUDRAGIT™ RL/RS mixtures are insoluble in water and in digestive fluids; however, coatings formed from them are swellable and permeable in aqueous solutions and digestive fluids.

The EUDRAGIT™ RL/RS dispersions may be mixed together in any desired ratio in order to ultimately obtain a controlled-release formulation having a desirable dissolution profile. Desirable controlled-release formulations may be obtained, for instance, from a retardant coating derived from one of a variety of coating combinations, such as 100% EUDRAGIT™ RL; 50% EUDRAGIT™ RL and 50% EUDRAGIT™ RS; or 10% EUDRAGIT™ RL and EUDRAGIT™ 90% RS. Of course, one skilled in the art will recognize that other acrylic polymers may also be used, for example, others under the EUDRAGIT™ brand. In addition to modifying the dissolution profile by altering the relative amounts of different acrylic resin lacquers, the dissolution profile of the ultimate product may also be modified, for example, by increasing or decreasing the thickness of the retardant coating.

The stabilized product may be obtained by subjecting the coated substrate to oven curing at a temperature above the Tg (glass transition temperature) of the plasticized acrylic polymer for the required time period, the optimum values for temperature and time for the particular formulation being determined experimentally. In certain embodiments of the present invention, the stabilized product is obtained via an oven curing conducted at a temperature of about 45° C. for a time period from about 1 to about 48 hours. It is also contemplated that certain products coated with the controlled-release coating of the present invention may require a curing time longer than 24 to 48 hours, e.g., from about 48 to about 60 hours or more.

The coating solutions preferably contain, in addition to the film-former, plasticizer, and solvent system (i.e., water), a colorant to provide elegance and product distinction. Color may be added to the solution of the compositions of the invention instead of, or in addition to the aqueous dispersion of hydrophobic material. For example, color may be added to an AQUACOAT™ product via the use of alcohol or propylene glycol based color dispersions, milled aluminum lakes and opacifiers such as titanium dioxide by adding color with shear to the water soluble polymer solution and then using low shear to the plasticized AQUACOAT™ product.

Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retardant effect of the coating.

Spheroids or beads coated with the compositions of the invention can be prepared, for example, by dissolving the compositions of the invention in water and then spraying the solution onto a substrate, for example, non pareil 18/20 beads, using a Wuster insert. Optionally, additional ingredients can also be added prior to coating the beads in order to assist the binding of the compositions of the invention to the beads, and/or to color the solution, etc. For example, a product which includes hydroxypropyl methylcellulose with or without colorant (e.g., OPADRY™ product, commercially available from Coloron, Inc.) may be added to the solution and the solution mixed (e.g., for about 1 hour) prior to application onto the beads. The resultant coated substrate, beads in this example, may then be optionally overcoated with a bather agent to separate the compositions of the invention from the hydrophobic controlled release coating. An example of a suitable barrier agent is one which comprises hydroxypropyl cellulose. However, any film-former known in the art may be used. It is preferred that the barrier agent does not affect the dissolution rate of the final product.

Immediate release particles according to the present invention may be coated with a controlled release coating in order to change the release rate to obtain the dissolution rates according to the present invention.

Press Coated, Pulsatile Dosage Form

In another embodiment of the present invention, the compositions of the invention can be administered via a press coated pulsatile drug delivery system suitable for oral administration with a controlled release component, which contains a compressed blend of an active agent (e.g., the components of the compositions of the invention) and one or more polymers, substantially enveloped by an immediate release component, which contains a compressed blend of the active agent and hydrophilic and hydrophobic polymers. The immediate-release component preferably comprises a compressed blend of active agent and one or more polymers with disintegration characteristics such that the polymers disintegrate rapidly upon exposure to the aqueous medium.

The controlled-release component preferably can comprise a combination of hydrophilic and hydrophobic polymers. In this embodiment, once administered, the hydrophilic polymer will dissolve away to weaken the structure of the controlled-release component, and the hydrophobic polymer will retard the water penetration and help to maintain the shape of the drug delivery system.

In accordance with the present invention, the term “polymer” includes single or multiple polymeric substances, which can swell, gel, degrade or erode on contact with an aqueous environment (e.g., water). Examples include alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polacrilin potassium, powdered cellulose, pregelatinized starch, sodium alginate, sodium starch glycolate, starch, ethylcellulose, gelatin, hydroxyethyl cellulose, hydroxypropyl cellulose, hydroxypropyl methylcellulose, polymethacrylates, povidone, pregelatinized starch, shellac, and zein, and combinations thereof.

The term “hydrophilic polymers” as used herein includes one or more of carboxymethylcellulose, natural gums such as guar gum or gum acacia, gum tragacanth, or gum xanthan, hydroxyethyl cellulose, hydroxypropyl cellulose, hydroxypropyl methylcellulose, methylcellulose, and povidone, of which hydroxypropyl methylcellulose is further preferred. The term “hydrophilic polymers” can also include sodium carboxymethylcellulose, hydroxymethyl cellulose, polyethelene oxide, hydroxyethyl methyl cellulose, carboxypolymethylene, polyethelene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), an alkali metal or alkaline earth metal, carageenate alginates, ammonium alginate, sodium alganate, or mixtures thereof.

The hydrophobic polymer of the drug delivery system can be any hydrophobic polymer which will achieve the goals of the present invention including, but not limited to, one or more polymers selected from carbomer, carnauba wax, ethylcellulose, glyceryl palmitostearate, hydrogenated castor oil, hydrogenated vegetable oil type 1, microcrystalline wax, polacrilin potassium, polymethacrylates, or stearic acid, of which hydrogenated vegetable oil type 1 is preferred. Hydrophobic polymers can include, for example, a pharmaceutically acceptable acrylic polymer, including, but not limited to, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methyl methacrylate) copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. The polymers may also be pH dependent.

The present invention also provides a method for preparing a press coated, pulsatile drug delivery system comprising the compositions of the invention suitable for oral administration. This method can include the steps of combining an effective amount of the components of the compositions of the invention, or a pharmaceutically acceptable salt thereof, and a polymer to form an immediate-release component; combining an effective amount of an active agent (e.g., the components of the compositions of the invention), or a pharmaceutically acceptable salt thereof, and a combination of hydrophilic and hydrophobic polymers to form a controlled release component; and press coating the controlled-release component to substantially envelop the immediate release component.

A preferred embodiment further can include the steps of combining an effective amount of an active agent (e.g., the components of the compositions of the invention), or a pharmaceutically acceptable salt thereof, and a polymer to form an immediate release component, and press coating the immediate release component to substantially envelop the controlled release component. In another preferred embodiment, the combining steps can be done by blending, wet granulation, fluid-bed granulation, or dry granulation according to methods recognized in the art.

The dosage form of the invention may be administered to mammalian subjects, including: humans, monkeys, apes, dogs, cats, cows, horses, rabbits, pigs, mice and rats.

The dosage form of the invention may be administered orally (e.g., in liquid form within a solvent such as an aqueous or non-aqueous liquid, or within a solid carrier). Administration can be performed daily, weekly, monthly, every other month, quarterly or any other schedule of administration as a single dose injection or infusion, multiple doses, or in continuous dose form. The administration of the pharmaceutical compositions of the present invention can be intermittent or at a gradual, continuous, constant or controlled rate to a subject. In addition, the time of day and the number of times per day that dosage form(s) is administered can vary.

For parenteral administration, in one embodiment, the agents of the invention can be formulated generally by mixing it at the desired degree of purity, in a unit dosage injectable form (solution, suspension, or emulsion), with a pharmaceutically acceptable carrier(s) described above.

Any dosage form used for therapeutic administration should be sterile. Sterility can readily be accomplished by filtration through sterile filtration membranes (e.g., 0.2 micron membranes). Therapeutics generally are placed into a container having a sterile access port, for example, an intravenous solution bag or vial having a stopper pierceable by a hypodermic injection needle.

The appropriate dose of the compound will be that amount effective to prevent occurrence of amoebiasis. By “effective amount”, “therapeutic amount” or “effective dose” is meant that amount sufficient to elicit the desired pharmacological or therapeutic effects, thus resulting in effective prevention or treatment of the disorder or condition.

In a further embodiment, the present invention provides kits (i.e., a packaged combination of reagents with instructions) containing the active agents of the invention useful for treating amoebiasis (including other parasitic infections).

The kit can contain a pharmaceutical composition that includes one or more agents of the invention effective for treating amoebiasis and an acceptable carrier or adjuvant, e.g., pharmaceutically acceptable buffer, such as phosphate-buffered saline, Ringer's solution or dextrose solution.

It may further include other materials desirable from a commercial and user standpoint, including other buffers, diluents, filters, needles, syringes, and package inserts with instructions for use.

The agents may be provided as dry powders, usually lyophilized, including excipients that upon dissolving will provide a reagent solution having the appropriate concentration.

The kit comprises one or more containers with a label and/or instructions. The label can provide directions for carrying out the preparation of the agents for example, dissolving of the dry powders, and/or treatment for e.g. amoebosis.

The label and/or the instructions can indicate directions for in vivo use of the pharmaceutical composition. The label and/or the instructions can indicate that the pharmaceutical composition is used alone, or in combination with another agent to treat e.g., amoebosis.

The label can indicate appropriate dosages for the agents of the invention as described supra.

Suitable containers include, for example, bottles, vials, and test tubes. The containers can be formed from a variety of materials such as glass or plastic. The container can have a sterile access port (for example the container can be an intravenous solution bag or a vial having a stopper pierceable by a needle such as a hypodermic injection needle).

The following examples are provided to further illustrate aspects of the invention. These examples are non-limiting and should not be construed as limiting any aspect of the invention.

### EXAMPLE

### Example 1

Results

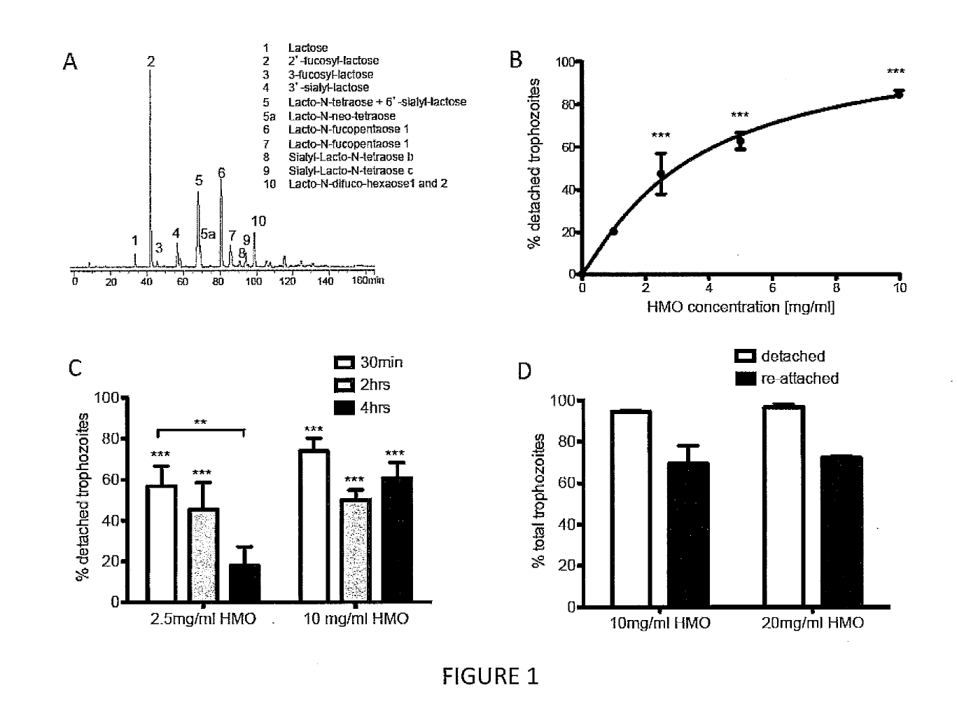

HMO detach E. histolytica trophozoites. HMO detach E. histolytica trophozoites in a dose dependent manner (FIG. 1A). The physiologic concentration of HMO (10 mg/ml) detached 80% of trophozoites within 30 min incubation. Light microscopic observations show that detached trophozoites round up and float in clusters. However, HMO did not change the osmolarity of the media, nor did it kill trophozoites and 98.1±2.0% trophozoites are viable after 2 hr incubation with 10 mg/ml HMO versus 93.8±2.6% in the untreated control. With this high concentration of HMO, trophozoites detachment remains constant over the 4 hr time period tested (FIG. 1B). However, long incubations with low concentrations of HMO (2.5 mg/ml) result in less detachment compared to the shorter incubations (FIG. 1B). This suggests that the HMO concentration reduce over time and that HMO mediated detachment might be reversible. Complete removal of HMO resulted in re-attachment of the majority of trophozoites, confirming this hypothesis (FIG. 1C).

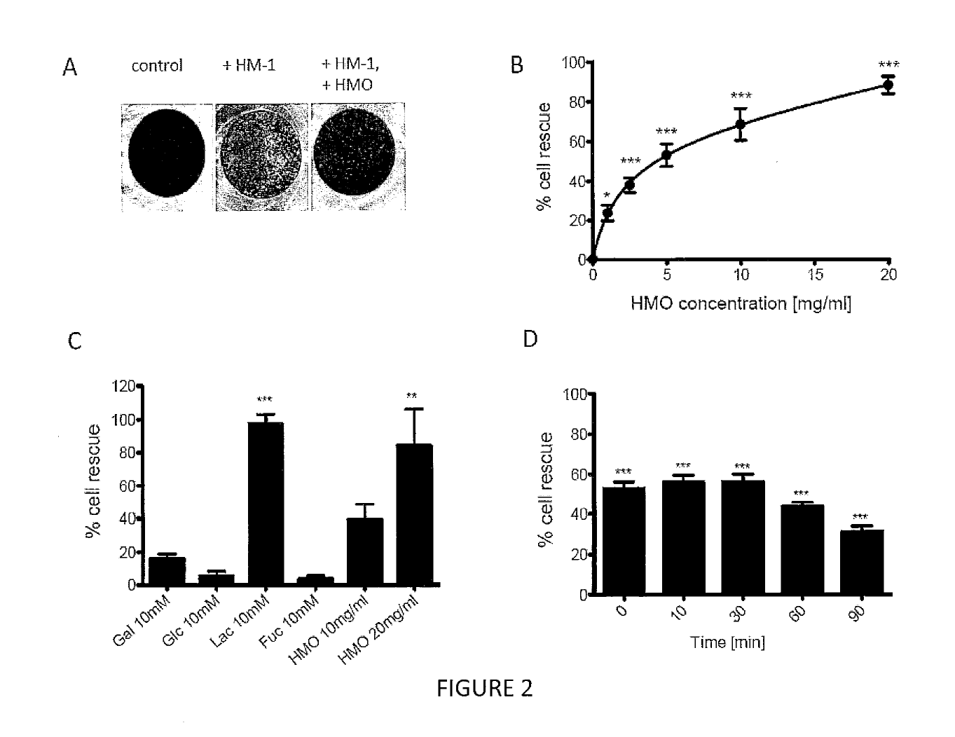

HMO prevent enterocyte cytotoxicity by E. histolytica in in vitro cocultures. Previous studies have shown reduced Entamoeba attachment and cytotoxicity in the presence of selected carbohydrates (Cano-Mancera and Lopez-Revilla, 1987; Ravdin and Guerrant, 1981; Bracha and Mirelman, 1984). We tested the effect of HMO on Entamoeba induced cytotoxicity in in vitro cocultures with HT-29 cell layers and calculated the % cell rescue (FIG. 2A). When HMO were present at the start of coculture, we found that the protective effect of HMO is dose dependent and that 20 mg/ml HMO rescued 88±4.4% of the cell layer (FIG. 2B). The cell protective effect of 20 mg/ml HMO is similar to the effect of 10 mM lactose and better than the effect of 10 mM galactose (FIG. 2C). Fucose and glucose do not prevent cytotoxicity (FIG. 2C) (Cano-Mancera and Lopez-Revilla, 1987). To test if HMO still had a cytoprotective effect on established cocultures, HMO was added at various time points after the start of coculture and cytotoxicity/cell rescue was measured after 2 hrs. We found that HMO protect cell layers from the cytotoxic effect of trophozoites after the onset of cytotoxicity (FIG. 2D). Treatment with 20 mg/ml after 90 min of cocultures could still rescue 31±4.7% of the cell layer. These data suggest that HMO can prevent both the initiation and progression of Entamoeba induced host cell cytotoxicity.

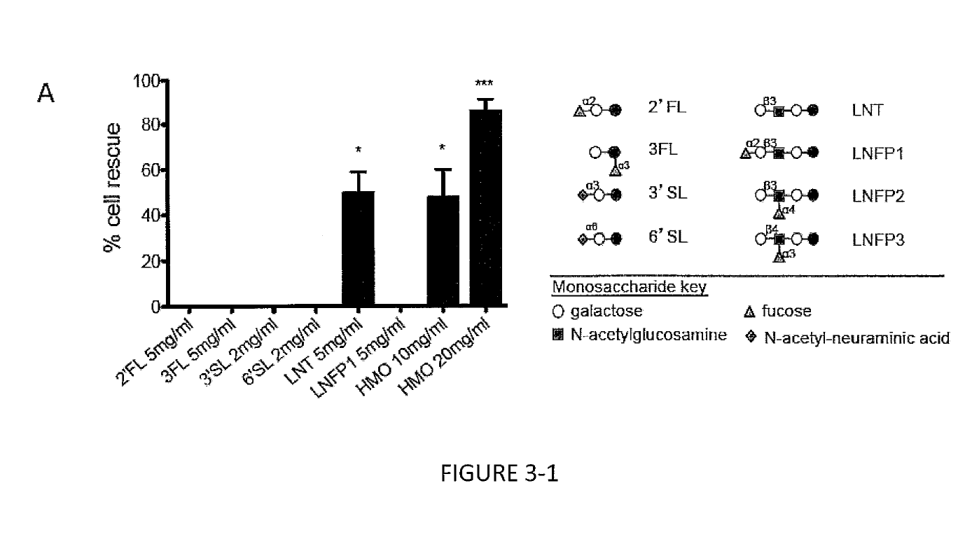

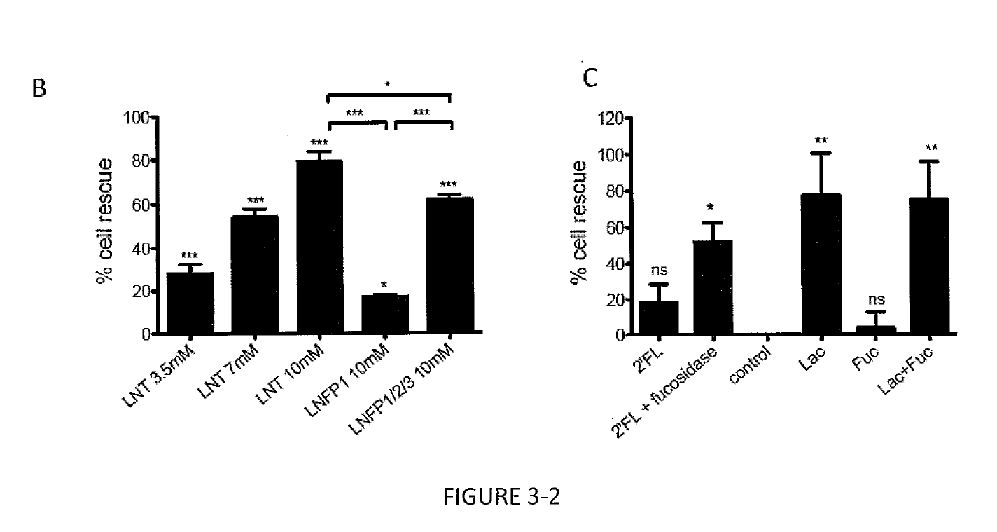

Individual HMO prevent enterocyte cytotoxicity by E. histolytica in in vitro co-cultures. HMO are a complex mixture of structurally different oligosaccharides. To assess which of the individual HMO are effective in the cytotoxicity assay we tested six commercially available HMO standards in physiologically relevant concentrations (FIG. 3A). Out of the individual HMO tested only Lacto-N-tetraose (LNT), significantly protected the cell layer in coculture with trophozoites. 5 mg/ml LNT, a neutral unsubstituted tetrasaccharide, rescued 49.5±9.2% of the cell layer. This is similar to the effect of 10 mg/ml pooled HMO (47.5%±12.2). Remarkably, physiological concentrations of the fucosylated forms of lactose: 2′fucosyl-lactose (2′FL) and 3-fucosyl-lactose (3FL), as well as the sialylated forms 3-sialyl-lactose (3′SL) and 6-sialyl-lactose (6′SL) failed to prevent monolayer destruction. In contrast to the strong effect of 5 mg/ml LNT, 5 mg/ml LNFP1 (5.8 mM), had no effect in rescuing enterocytes. Since the only structural difference between these HMO is a fucose residue on the terminal Gal of LNFP1, these findings suggest that the terminal Gal residue in the HMO structure is required for its cytoprotective effect. To further investigate this hypothesis we tested equimolar concentrations of LNT and LNFP1 in the cytotoxicity assay. LNT had a dose-dependent effect reaching 79±8.3% cell rescue at 10 mM, whereas the effect of equimolar concentration of LNFP1 was significantly lower (16.9±1.7%)(FIG. 3B). Interestingly, a 10 mM mixture of the isomers LNFP1, LNFP2 and LNFP3 consisting of 62% LNFP1 had a significantly higher effect (61.7±5.5%) than 10 mM pure LNFP1. This suggests that LNFP2 and/or LNFP3 strongly inhibit trophozoite mediated cytotoxicity. Both LNFP2 and 3 are substituted by fucose at the subterminal N-acetyl-glucosamine (GlcNAc) residue leaving the terminal Gal accessible (insert in FIG. 3A). This supports our hypothesis that the terminal Gal is required for the cytoprotective effect of HMO. If this is true, removal of Fuc from terminal Gal of HMO should restore the protective effect. Therefore we treated 2′FL with a specific alpha-1,2 fucosidase and compared its effect with untreated 2′FL, Lac, Fuc and with a mixture of Lac and Fuc. Similar to the mixture of equimolar concentrations of Lac and Fuc, fucosidase treatment of 2′FL resulted in significant rescue of the cell layer of 51.7±10.76% (FIG. 3C). This finding strongly suggests that not the presence of Fuc per se but the blockage of the terminal Gal renders 2′FL ineffective.

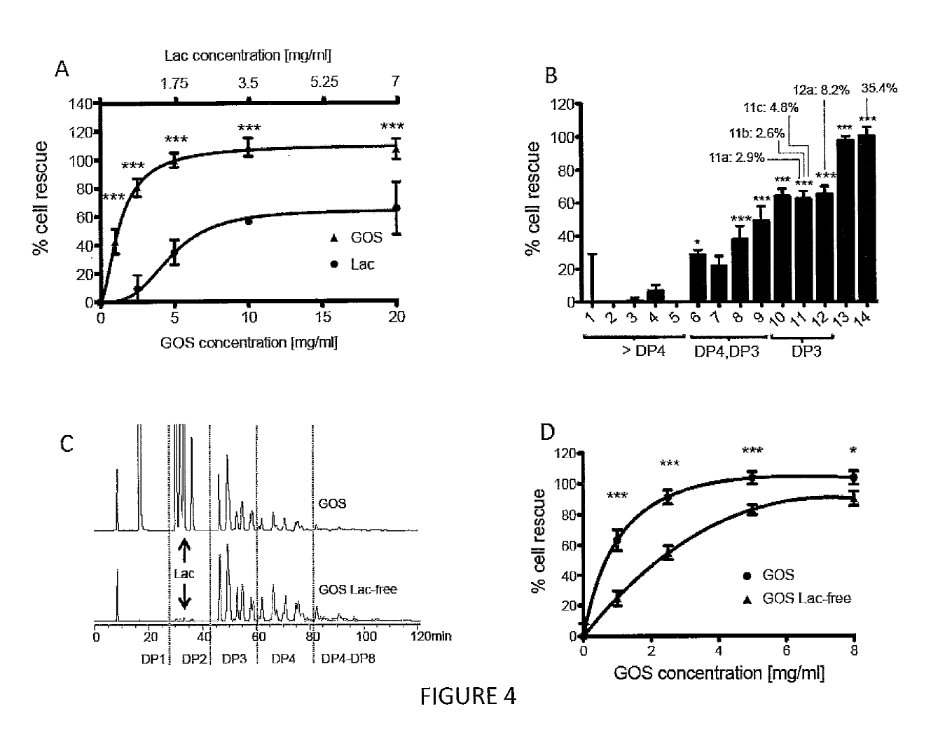

GOS prevent E. histolytica cytotoxicity independent of its lactose content. Galactooligosaccharides (GOS), which are currently widely added to infant formula as aprebiotic, consist of varying numbers of Gal residues attached to a single Glc at the reducing end. Since our data indicates that terminal Gal is essential for the cytoprotective effect of HMO, we tested whether GOS are also effective against E. histolytica cytotoxicity. We found that GOS indeed protect cell monolayers from destruction by trophozoites in a dose-dependent manner (FIG. 4A), with 5 mg/ml being fully protective. To rule out that this effect is due to Lac, we tested the respective Lac concentrations present in each GOS sample on Entamoeba mediated cytotoxicity. The effect of Lac alone was significantly lower than the effect of lactose containing GOS, suggesting that GOS contains other oligosaccharides that prevent cytotoxicity (FIG. 4A). To prove this hypothesis, we separated GOS by size exclusion chromatography and determined the lactose content and cytoprotective effect of the resulting 14 fractions. Consistent with their high lactose content, fractions 13 and 14 have a high cytoprotective effect on cocultures (FIG. 4B). Fractions 10-12 contain almost exclusively trisaccharides (DP3) and hardly any lactose and showed a significantly cytoprotective effect (FIG. 4B). Fractions 6-9 contain predominantly tetrasaccharides (DP4) and mediate lower but still significant cell rescue. Early eluted fractions containing larger oligosaccharides up to DP8 had no effect on cytotoxicity, which can be explained by the much lower content of oligosaccharides>DP4 in total GOS (FIG. 4C). We prepared lactose ‘free’ GOS (<0.5% lactose) by pooling all GOS fractions with a lactose content ≦3% (fractions 1-11b, 32 fractions) and compared its cytoprotective effect in the cytotoxicity assay to equal concentrations of lactose containing GOS (FIG. 4D). In accordance with the additional effect of GOS compared to Lac alone (FIG. 4A) we found a dose dependent cell rescue effect in the Lac-free GOS, indicating a lactose independent effect of GOS. Remarkably, at 8 mg/ml, the effect of Lac-free GOS reached 90.4±4.8% and was in the range of Lac containing GOS. Similar to HMO, the concentrations of GOS found in formula (8 mg/ml) are not toxic to trophozoites (96.6±1.3% viable compared to 93.8±2.6% in the control), detach trophozoites from glass (81.3±7.3% detachment within 30 min compared to control) and provide substantial protection of cell layers in established cocultures with E. histolytica trophozoites. These data confirm that the protective effect of GOS is lactose independent, results from a direct interaction with the trophozoites and is comparable to the effect of HMO.

1. A formulation comprising isolated lacto-N-tetraose (LNT) or variants, isomers, analogs and derivatives thereof and a pharmaceutically acceptable carrier.

2. A formulation comprising increased amounts of lacto-N-tetraose (LNT) or its equivalents, analogs and derivatives thereof.

3. (canceled)

4. (canceled)

5. The formulation of claim 2, wherein the pharmaceutical formulation is included in an infant formula, breast milk, baby food or nutritional supplement.

6. (canceled)

7. (canceled)

8. A method to prevent or treat a disease or disorder in a subject by administering human milk oligosaccharides (HMO) or galactooligosaccharides (GOS), or equivalents, analogs and derivatives thereof in an amount sufficient to treat and prevent the disease or disorder.

9. The method of claim 8, wherein the HMO is lacto-N-tetraose (LNT).

10. The method of claim 8, wherein the subject is a mammal.

11. (canceled)

12. (canceled)

13. The method of claim 10, wherein the mammal is a human, monkey, rat, mouse, dog, cat, pig, goat, sheep, horse or cow.

14. (canceled)

15. The method of claim 8, wherein the disease or disorder is a parasitic disease, gastrointestinal disease, amoebiasis, amoebic dysentery or amoebic colitis.

16. The method of claim 8, wherein the disease is associated with Entamoeba histolytica infection.

17. The method of claim 8, wherein the HMO or GOS, or equivalents, analogs and derivatives thereof are administered by means of infant formula, biscuits, adult nutritional supplements, dairy products, fruit drinks, acidic beverages, confectionery, cereal bars or baked goods.

18. The formulation of claim 1, wherein the formulation is a tablet or a caplet.

19. The formulation of claim 18, wherein the tablet or caplet is a multi-layered.

20. The formulation of claim 1, wherein the formulation is a pharmaceutical formulation.

21. The formulation of claim 18, wherein the tablet or caplet is a matrix tablet or caplet.

22. The formulation of claim 1, wherein the formulation is a multiparticulate formulation.

23. The formulation of claim 22, wherein the multiparticulates are encapsulated.

24. The formulation of claim 22, wherein the multiparticulates are pressed into a tablet.

25. (canceled)
